(19) United States
(12) Patent Application Publication
(10) Pub. No.: US 2025/0242171 A1
(43) Pub. Date: Jul. 31, 2025

(54) MINIMALLY INVASIVE AND ENDOSCOPIC IN-VIVO BIOPRINTING SYSTEMS AND METHODS

(71) Applicant: PhosPrint P.C., Athens (GR)

(72) Inventors: Ioanna Zergioti, Athens (GR); Apostolos Klinakis, Attica (GR); Theofilos Kotseroglou, Hillsborough, CA (US); Ioannis Theodorakos, Athens (GR)

(21) Appl. No.: 19/040,593

(22) Filed: Jan. 29, 2025

Related U.S. Application Data
(60) Provisional application No. 63/626,951, filed on Jan. 30, 2024.

(51) Int. Cl. A61N 5/06; A61K 35/34; A61M 25/10; A61N 5/067

(52) U.S. Cl. CPC A61N 5/062; A61K 35/34; A61M 25/104; A61N 5/0601; A61N 5/067; A61M 2025/105; A61M 2202/09; A61M 2210/125; A61N 2005/063

(57) ABSTRACT

A system may include a donor supply sub-system configured to supply donor material to a donor substrate. The system may include a material transfer sub-system comprising a laser configured to illuminate the donor substrate via a transfer beam. The system may include an instrument head of a medical instrument configured to house the donor substrate, wherein upon an illumination of the donor substrate, the donor material is transferred to receiver material in vivo.

The present application claims the benefit under 35 U.S.C. § 119(e) of U.S. Provisional App. No. No. 63/626,951 filed by Zergioti on Jan. 30, 2024, entitled “MINIMALLY INVASIVE AND ENDOSCOPIC IN-VIVO BIOPRINTING SYSTEMS AND METHODS”, which is incorporated herein by reference in its entirety.

### BACKGROUND

Many diseases in both humans and nonhumans involve a deficit in the function of organs within the body. This deficit of function is often brought on by a loss of functioning cells within the organ. If untreated, the loss of functioning cells may lead to organ failure and an aggravated disease state. Since most organs are internal to the body, the replacement of functional cells is difficult, particularly the replacement of cells without wholesale replacement of the organ, which requires complex surgeries and immunosuppressant chemotherapy. Therefore, there is a need for a system and method to replace cells in an organ without resorting to tissue or organ transplant.

### SUMMARY

In some embodiments, the techniques described herein relate to a system including: a donor supply sub-system configured to supply donor material to a donor substrate; a material transfer sub-system including a laser configured to illuminate the donor substrate via a transfer beam; and an instrument head of a medical instrument configured to house the donor substrate, wherein upon an illumination of the donor substrate, the donor material is transferred to receiver material in vivo.

In some embodiments, the techniques described herein relate to a system, wherein the medical instrument includes an endoscope.

In some embodiments, the techniques described herein relate to a system, wherein the instrument head includes at least one optical fiber configured to propagate the transfer beam; and a piezoelectric actuator coupled to the at least one optical fiber and configured to steer the at least one optical fiber.

In some embodiments, the techniques described herein relate to a system, wherein the instrument head further includes: an angled section; and an optical element configured to direct the transfer beam through the angled section.

In some embodiments, the techniques described herein relate to a system, wherein the instrument head includes a first fiber configured to propagate the transfer beam and a second fiber configured to propagate a photopolymerization beam.

In some embodiments, the techniques described herein relate to a system including at least one optical fiber array, wherein the at least one optical fiber array includes an array of micro-reservoirs configured to receive donor material, whereupon illumination by the transfer beam, the donor material is transferred from the array of micro-reservoirs.

In some embodiments, the techniques described herein relate to a system, wherein the instrument head further includes a fiber array configured to propagate the transfer beam to the array of micro-reservoirs.

In some embodiments, the techniques described herein relate to a system, wherein at least a portion of the instrument head is configured to rotate relative to the medical instrument.

In some embodiments, the techniques described herein relate to a system, wherein the instrument head includes two optical fiber arrays arranged orthogonal relative to each other.

In some embodiments, the techniques described herein relate to a system, further including a film rolling sub-system including; a donor material tank coupled to the donor supply sub-system; a film coating interface fluidly coupled to the donor material tank; and a film threaded within the film rolling sub-system and configured to receive a coating of donor material from the film coating interface, wherein upon movement of the film coated with the donor material to a print window, the film receives the transfer beam.

In some embodiments, the techniques described herein relate to a system, wherein the instrument head includes: a printing structure coupled to at least one of the donor supply sub-system and the material transfer sub-system, the printing structure including: an outer surface; a sensor layer including at least one sensor; and a fluidics layer capable of storing donor material.

In some embodiments, the techniques described herein relate to a system, wherein the instrument head includes a balloon structure.

In some embodiments, the techniques described herein relate to a system, further including at least one of a laser absorbing layer configured to absorb energy from the laser and release energy onto the donor material.

In some embodiments, the techniques described herein relate to a system wherein the donor material includes a laser-absorbing agent.

In some embodiments, the techniques described herein relate to a method for in-vivo cell replacement including: loading donor material onto a donor substrate; and printing the donor material from the donor substrate via an instrument head of a medical instrument onto receiver material in vivo via Laser Induced Forward Transfer (LIFT).

In some embodiments, the techniques described herein relate to a method, wherein the medical instrument includes an endoscope.

In some embodiments, the techniques described herein relate to a method, wherein the instrument head includes a balloon structure.

In some embodiments, the techniques described herein relate to a method, wherein the donor material includes cells.

In some embodiments, the techniques described herein relate to a method, wherein the donor material further includes at least one of hydrogels, growth factors, matrix proteins, and platelet-rich plasma.

In some embodiments, the techniques described herein relate to a method, wherein the cells include cardiac cells.

In some embodiments, the techniques described herein relate to a method, wherein the receiver material includes myocardium tissue.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

FIG. 1 illustrates a block diagram of a system for printing donor material onto receiver material, in accordance with one or more embodiments of the disclosure.

FIG. 2 illustrates a diagram of the bioprinting subsystem, in accordance with one or more embodiments of the disclosure.

FIG. 3 illustrates a medical instrument coupled to an instrument head for performing in vivo printing, in accordance with one or more embodiments of the disclosure.

FIG. 4A illustrates a cross-section side view of an instrument head, in accordance with one or more embodiments of the disclosure.

FIG. 4B illustrates a cross-section side view of an instrument head with an articulated, steerable, or angled section, in accordance with one or more embodiments of the disclosure.

FIG. 5 illustrates a side view of an instrument head, with a tip of the instrument head shown enlarged in an inset, in accordance with one or more embodiments of the disclosure.

FIG. 6A illustrates a system for printing donor material onto receiver material in accordance with one or more embodiments of the disclosure.

FIG. 6B illustrates a perspective view of an input of an optical fiber array, in accordance with one or more embodiments of the disclosure.

FIGS. 7A-7C illustrate views of an instrument head, in accordance with one or more embodiments of the disclosure.

FIG. 8A illustrates a perspective front view of an instrument head, in accordance with one or more embodiments of the disclosure.

FIG. 8B illustrates a close-up perspective view of an underside of the instrument head, in accordance with one or more embodiments of the disclosure.

FIGS. 9A to 9C illustrate a donor material delivery device of a balloon type, in accordance with one or more embodiments of the disclosure.

FIG. 10 illustrates a process flow diagram depicting a method for in-vivo cell replacement, in accordance with one or more embodiments of the disclosure.

### DETAILED DESCRIPTION

Before explaining one or more embodiments of the disclosure in detail, it is to be understood that the embodiments are not limited in their application to the details of construction and the arrangement of the components or steps or methodologies set forth in the following description or illustrated in the drawings. In the following detailed description of embodiments, numerous specific details may be set forth in order to provide a more thorough understanding of the disclosure. However, it will be apparent to one of ordinary skill in the art having the benefit of the instant disclosure that the embodiments disclosed herein may be practiced without some of these specific details. In other instances, well-known features may not be described in detail to avoid unnecessarily complicating the instant disclosure.

As used herein a letter following a reference numeral is intended to reference an embodiment of the feature or element that may be similar, but not necessarily identical, to a previously described element or feature bearing the same reference numeral (e.g., 1, 1a, 1b). Such shorthand notations are used for purposes of convenience only and should not be construed to limit the disclosure in any way unless expressly stated to the contrary.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of “a” or “an” may be employed to describe elements and components of embodiments disclosed herein. This is done merely for convenience and “a” and “an” are intended to include “one” or “at least one,” and the singular also includes the plural unless it is obvious that it is meant otherwise.

Finally, as used herein any reference to “one embodiment” or “some embodiments” means that a particular element, feature, structure, or characteristic described in connection with the embodiment is included in at least one embodiment disclosed herein. The appearances of the phrase “in some embodiments” in various places in the specification are not necessarily all referring to the same embodiment, and embodiments may include one or more of the features expressly described or inherently present herein, or any combination of sub-combination of two or more such features, along with any other features which may not necessarily be expressly described or inherently present in the instant disclosure.

Broadly, embodiments of the inventive concepts disclosed herein are directed to a method and a system for printing biomaterials into a subject (e.g., human or non-human) in vivo. The method and system may include a laser-induced forward transfer (LIFT) system. The system may include a donor substrate configured to bind donor material (e.g., cells or other biocompatible material) and a laser configured to focus a laser pulse on the donor substrate, which activates a transfer of the donor material from the donor substrate to a receiver material (e.g., living tissue). The system may include a housing configured to house the donor substrate within an insertion section of an endoscope, or other instrument, such as a balloon configured to house the donor substrate and the laser. The method may include uses of the system for the treatment of various diseases, such as cardiomyopathy.

Certain device and system implementations disclosed in herein above can be positioned within a body cavity of a subject (in vivo”), or a portion of the device can be placed within the body cavity, in combination with a positioning system such as any of the embodiments disclosed or contemplated herein. An “in vivo device” as used herein means any device that can be positioned, operated, or controlled at least in part by a user while being positioned within a body cavity of a patient, including any device that is coupled to a support component such as a rod, tube, body, or other such components that is disposed through an opening or orifice of the body cavity, also including any device positioned substantially against or adjacent to a wall of a body cavity of a patient, further including any such device that is internally actuated (having no external source of motive force), and additionally including any device that may be used laparoscopically or endoscopically during a surgical procedure.

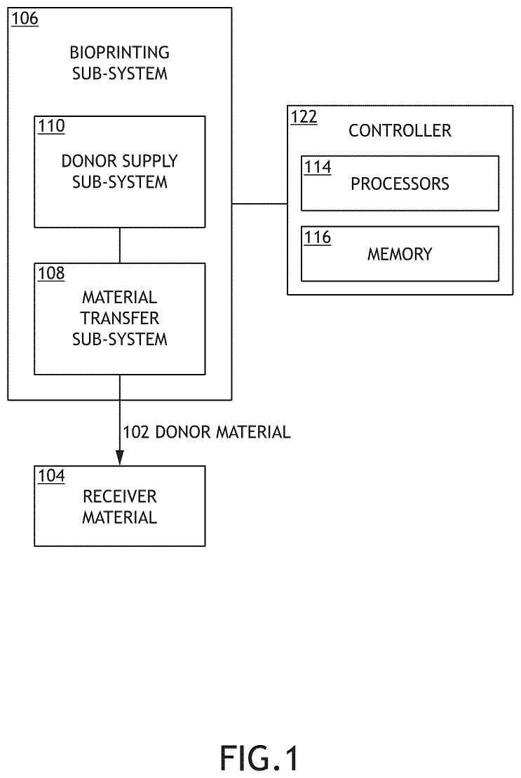

Referring now to FIGS. 1-10, various non-limiting examples of bioprinting using the systems and methods disclosed herein are described in greater detail.

FIG. 1 illustrates a block diagram of a system 100 for printing donor material 102 onto receiver material 104, in accordance with one or more embodiments of the disclosure.

The receiver material 104 may be any tissue, organ, organoid, graft, or other material that may be a substrate for tissue engineering and transplantation. For example, the receiver material 104 may include cells or tissues within an organism. For instance, the receiver material 104 may include tissues, organs, organoids, matrices, or another biomaterial within a mammal, such as a human subject. In another example, the receiver material may include biocompatible materials including, but not limited to, prosthetics, implants, and other internal medical devices (e.g., heart valves). The receiver material 104 may also include other biological tissues, including but not limited to, smooth muscle tissue, skeletal muscle tissue, blood vessels, skin, bone, connective tissue (e.g., facia), epithelial tissue, heart tissue, and nervous tissue. For example, the receiver material may include any section of the gastrointestinal tract (e.g., stomach), the diaphragm, the uterus or fallopian tubes. In another example, the receiver material may include bladder tissue, cartilaginous tissue or esophageal tissue.

The donor material 102 may be any living or biocompatible material that is to be deposited onto the receiver material 104, including but not limited to a bioink, a hydrogel, a cell, a tissue, an organoid, a protein, a nucleic acid, an extracellular material (e.g., matrices), an intracellular material, or a scaffolding material. For example, the donor material 102 may be a urothelial cell, a fibroblast, a mesenchymal cell, an adipocyte, a keratinocyte (e.g., esophageal keratinocytes), a chondrocyte an immune cell, a muscle cell, a nerve cell, an insulinogenic cell a cardiomyocyte, or a stem cell. For instance, the donor material may be urothelial cells that were harvested from bladder tissue or bladder-like tissues (e.g., urothelial cells scraped from a portion of a bladder, ureter, urethra, or renal pelvis). In another instance, the cell may be an insulinogenic B. In another instance, the cell may be an intestinal epithelial cell.

It should be understood that the donor material 102 may be derived from the recipient of the tissue transplant (e.g., an autologous transplant), or from another party (e.g., a heterologous transplant). It should also be understood that the cellular portions of the donor material may be derived, differentiated, or otherwise isolated from primary or non-primary sources. For example, the donor material 102 may be a progenitor cell (i.e., a cell having non-proliferative or low-proliferative qualities). In another example, the donor material 102 may be a stem cell, having high proliferative and/or differentiating capacity. For instance, the donor material 102 may comprise unipotent stem cells capable of producing urothelial cells. In particular, unipotent urothelial cells may be isolated from a bladder, a ureter, a urethra, or a renal pelvis, expanded in vitro, and then transferred to the receiver material 104.

In another example, the donor material 102 may comprise multipotent stem cells, capable of differentiating into more than one cell type. For instance, the donor material may include endoderm stem cells, or stem cells arising from an endoderm lineage. In particular, multipotent mesenchymal cells (e.g., derived from hemopoietic or adipose tissue) may be expanded and differentiated towards a urothelial fate. The resultant urothelial cells may then be transferred to the receiver material via the transfer beam 202. In another example, the donor material 102 may include mesoderm stem cells or stem cells arising from a mesoderm lineage. In another example, the donor material 102 may include ectoderm stem cells or stem cells arising from an ectoderm lineage. In other words, stem cells may arise from a mesodermal, endodermal, and or ectodermal origin, and may come from a common host.

In another example, the donor material 102 may comprise pluripotent stem cells capable of producing endodermic, mesodermic, or ectodermic lineages of cells. For instance, cells from the patient may be induced to become induced pluripotent stem cells (iPS). The resultant iPS cells are then expanded and differentiated into urothelial cells, which may then be transferred to the receiver material via the transfer beam 202.

In embodiments, the system 100 includes a bioprinting sub-system 106. The bioprinting sub-system 106 may include a material transfer sub-system 108 configured to direct the donor material 102 to the receiver material 104, and a donor supply sub-system 110 configured to supply donor material to the material transfer sub-system 108. The system 100 may further include a controller 112 communicatively coupled to the bioprinting sub-system 106. In embodiments, the controller 112 includes one or more processors 114 and memory 116. For example, the memory 116 may maintain program instructions configured to cause the one or more processors 114 to carry out any of the one or more process steps described throughout the present disclosure.

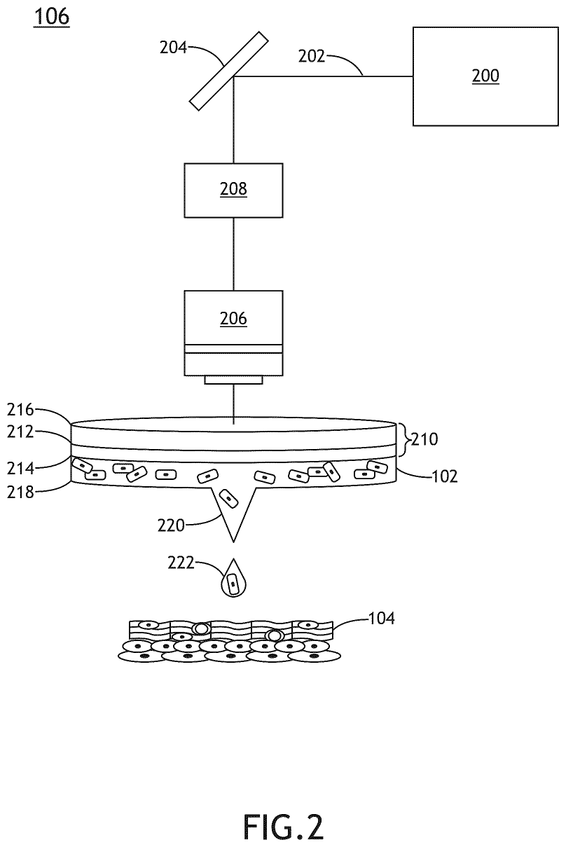

FIG. 2 illustrates a diagram of the bioprinting subsystem 106, in accordance with one or more embodiments of the disclosure. In embodiments, bioprinting subsystem 106 includes a transfer laser 200. The transfer laser 200 produces a transfer beam 202 that transfers donor material 102 to the receiver material 104.

The transfer laser 200 may be any laser known in the art used for transferring donor material 102, including but not limited to, a solid-state laser, a gas laser, a dye laser, or a semiconductor laser. For example, the transfer laser 200 may be a diode pumped solid state laser. For instance, the transfer laser 200 may be a diode pumped Nd: YAG solid-state micro-laser.

The transfer beam 202 produced by the transfer laser 200 may be of any wavelength or wavelength range known in the art. For example, the transfer laser 200 may produce a transfer beam 202 in the visible spectrum (e.g., 380 to 780 nm). For instance, the transfer beam 202 may have a wavelength of approximately 532 nm. In another example, the transfer laser 200 may produce a transfer beam 202 in the near infrared spectrum (e.g., 780 to 2500 nm).

In some embodiments, the transfer beam 202 produced by the transfer laser 200 may be pulsed. The pulse rate of the transfer beam 202 may be any pulse rate or range of pulse rates known in the art. For example, the transfer laser 200 may produce a transfer beam 202 with a pulse rate ranging from 1 Hz to 10 kHz. In another example, the transfer laser 200 may produce a transfer beam 202 with a pulse rate ranging from 10 Hz to 1 kHz. For instance, the transfer laser may produce a translation beam 202 with a pulse rate of approximately 10 Hz. In another example, the transfer laser 200 may produce a translation beam 202 with a pulse rate ranging from 100 Hz to 1 kHz. For instance, the transfer laser 200 may produce a translation beam 202 with a pulse rate of approximately 1 kHz.

In embodiments, the transfer laser 200 produces a pulsed transfer beam 202 with a specific pulse length or range of pulse lengths. The pulse length of the transfer beam 202 may be any pulse rate known in the art. For example, the length of the pulse of the transfer beam 202 may range from 60 ps to 6 ns. In another example, the length of the pulse of the transfer beam 202 may range from 100 ps to 1 ns. In another example, the length of the pulse of the transfer beam 202 may be approximately 600 ps.

In embodiments, the transfer laser 200 produces a transfer beam 202 with a specific fluence or range of fluences. The fluence of the transfer beam 202 may be any range or value known in the art. For example, the fluence of the transfer beam 202 may range from 10 mJ/cm² to 10 J/cm². In another example, the fluence of the transfer beam 202 may range from 100 mJ/cm² to 1 J/cm². In another example, the fluence of the transfer beam 202 may range from 100 mJ/cm² to 500 mJ/cm². In still another example, the fluence of the transfer beam 202 may range from 300 mJ/cm² to 800 mJ/cm².

In embodiments, the bioprinting sub-system 106 includes one or more optical elements configured to direct and/or focus the transfer beam 202. The optical elements may be any known in the art including, but not limited to, mirrors, lenses, and beamsplitters. For example, the one or more optical elements may include the one or more reflecting mirrors 204. In another example, the optical element may include one or more focusing lenses 206 (e.g., an objective lens). The focusing lens 206 may be any type of lens known in the art including, but not limited to, an achromatic lens. For example, the focusing lens may be an achromatic lens size to fit within an endoscope tool. In another example, the optical element may include a fixed attenuator plate.

In embodiments, the transfer laser assembly includes a donor substrate 210. The donor substrate 210 aids in the transfer of the donor material 102 to the receiver material 104. The donor substrate 210 may include a front surface 212. The front surface 212 faces the receiver material 104 and may be coated with a laser-absorbing layer 214 (e.g., a dynamic release layer (DRL)) that absorbs laser energy. The donor substrate 210 may further include a back surface 216 that initially receives the transfer beam 202. During LIFT, a suspension 218 containing donor material 102 may be coated over the laser-absorbing layer 214. When the transfer laser 200 is activated, the transfer beam 202 enters the back surface 216 of the donor substrate 210. Once the transfer beam 202 reaches the laser absorbing layer 214, localized heating at the laser absorbing layer 214 and the suspension 218 creates a high-pressure vapor bubble 220 within the localized area of the suspension 218. The expansion of the vapor bubble 220 then drives the ejection of a droplet 222 of the suspension 218 towards the receiver material 104.

In embodiments, the donor material 102 includes a laser-absorbing agent that acts as a laser-absorbing layer 214 or DRL. For example, donor material 102 that includes a laser-absorbing agent may, upon illumination from the transfer laser 200, cause the creation of a high-pressure vapor bubble 220 within the localized area of the suspension 218 that drives the ejection of a droplet 222 of the suspension 218 of the donor material towards the receiver material 104. Laser-absorbing agents include, but are not limited to, blood products and dyes. For example, dyes that can act as laser-absorbing agents include, but are not limited to, Allura Red AC (E129), Amaranth (E123), Carmoisine (E122), and Ponceau 4R (E124).

The size or range of sizes of the droplet 222 may be adjusted for the specific LIFT requirements. For example, the droplet diameter may range from 10 μm to 1 mm. In another example, the droplet diameter may range from 50 μm to 200 μm.

In embodiments, the system 100 includes a medical instrument 300 or a portion of a medical instrument 300. For example, the medical instrument 300 may include an instrument head 302 capable of printing donor material 102 in vivo. The medical instrument 300 may be configured as a hand-held instrument (e.g., an endoscope) or a tethered instrument, such as systems that include balloon-type instrument heads for performing angioplasty.

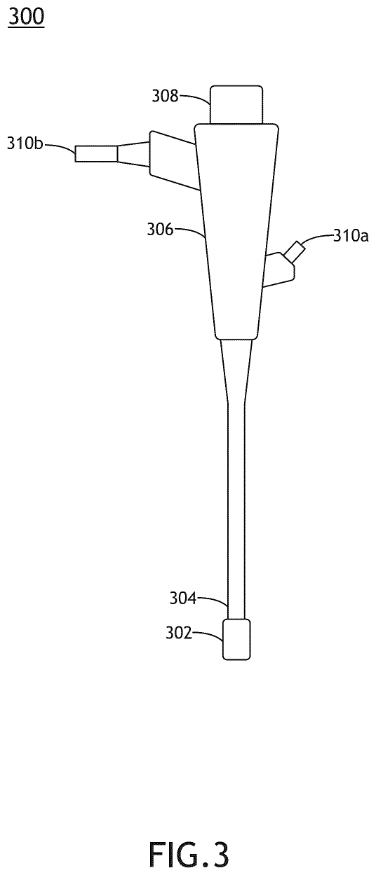

FIG. 3 illustrates a medical instrument 300 (e.g., an endoscope) coupled to an instrument head 302 (e.g., an endoscope head) for performing in vivo printing, in accordance with one or more embodiments of the disclosure. The instrument head 302 may be coupled to a tool end 304 of the medical instrument 300. The medical instrument 300 may include any type of medical instrument including, but not limited to, an endoscope. For example, the medical instrument 300 may include or be integrated with a gastroscope, colonoscope, bronchoscope, cystoscope, laparoscope, arthroscope, hysteroscope, laryngoscope, ureteroscope, nasopharyngoscope, or enteroscope. The medical instrument 300 may include an endoscope having any type of tool including, but not limited to, a camera, biopsy forceps, graspers, snares, scissors, electrocautery probes, coagulation probes, needles, suction devices, irrigation devices, stents, balloon dilators, guidewires, laser probes, trocars, clips, suture passers, and ultrasound probes. In another example, the medical instrument 300 includes an endoscope that includes only the instrument head 302. In another example, the medical instrument 300 includes an endoscope that includes only the instrument heads and 302 and a camera. In embodiments, the medical instrument 300 includes a handle 306 for grasping the medical instrument 300 during printing.

In embodiments, the medical instrument 300 may include an actuator 308 for switching on and/or controlling the bioprinting capabilities of the medical instrument 300. For example, the actuator 308 may be configured as a trigger, button, or dial.

In embodiments, the medical instrument 300 includes one or more ports 310a, 310b. For example, the medical instrument 300 may include a port 310a for powering the instrument head 302 or for supplying light (e.g., light for transfer, imaging, or photopolymerization). In another example, the medical instrument 300 may include a donor material port 310b for supplying donor material 102 to the instrument head 302.

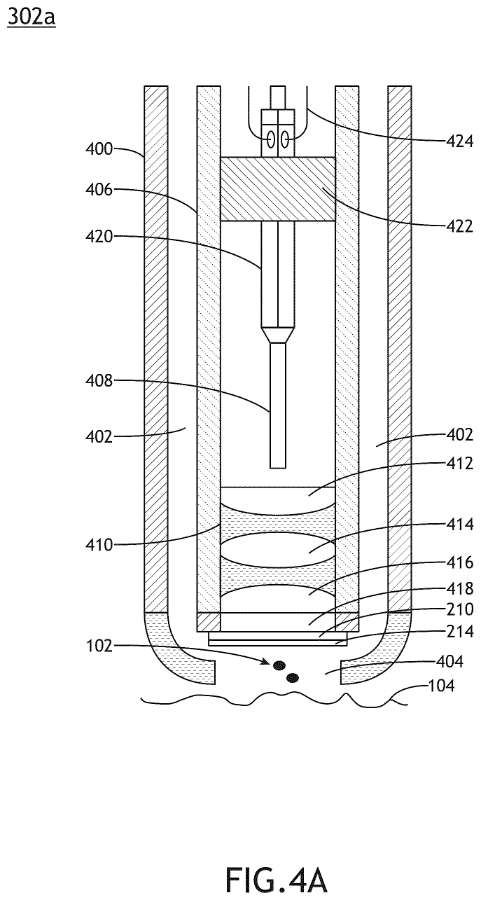

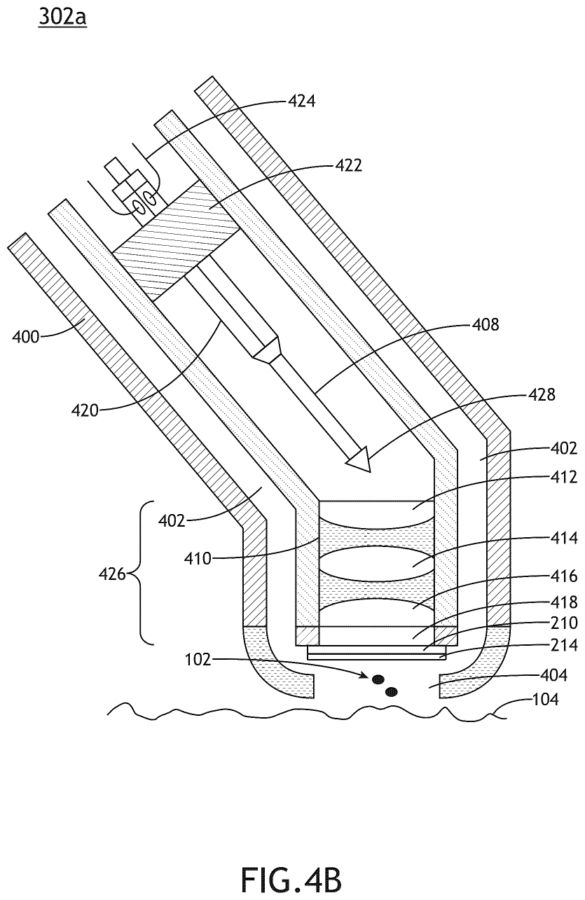

FIG. 4A illustrates a cross-section side view of an instrument head 302a, in accordance with one or more embodiments of the disclosure. The instrument head 302a utilizes a piezoelectric-actuating movement in printing the donor material 102 onto the receiver material 104. The instrument head 302a includes a housing 400 that houses the components of the instrument head.

In embodiments, the instrument head 302a includes a donor material path 402 that provides a pathway for donor material 102 to travel from a reservoir within or external to the instrument to an aperture 404 where the donor material 102 is printed onto the receiver material 104. The donor material path 402 be configured as a circulating path, where donor material 102 not immediately printed may be circulated away from the aperture (e.g., via the donor material path), or as a noncirculating path, where all donor material 102 entering the donor material path 402 is intended to be printed through the aperture. In embodiments, the instrument head 302a includes a sub-housing 406 configured to separate the donor material 102 from other instrument head componentry, including LIFT components.

In embodiments, the instrument head 302a includes an optical fiber 408 (e.g., acting as an excited cantilever) configured to deliver the transfer beam 202 to a lens set 410. The lens set 410 includes one or more optical elements 412, 414, 416 that focus and shape the transfer beam 202. The instrument head 302a may further include a donor substrate 210 (e.g., polyimide, glass, transparent plastic sheet) downstream of the lens set 410 and a laser absorbing layer 214 (e.g., a gold or nickel coating) disposed on the donor substrate. The instrument head 302a may further include an air gap 418 or matching fluid situated between the lens set 410 and the donor substrate 210.

In embodiments, the instrument head includes a piezoelectric actuator 420 (e.g., a piezoelectric actuator) coupled to the optical fiber 408 and configured to control or steer the movements of the optical fiber 408, assisting the transfer of the donor material 102 to the receiver material 104. The piezoelectric actuator 420 is coupled to a piezo mount 422, which is in turn coupled to receptacle 424, which supplies the necessary electrical power.

FIG. 4B illustrates a cross-section side view of an instrument head 302a with an articulated, steerable, or angled section 426, in accordance with one or more embodiments of the disclosure. For example, the section 426 may be articulated, steered manually or by automated means. In another example, the section 426 may be fixed. The instrument head 302a may further include an optical element 428 (e.g., a prism or mirror), that directs the transfer beam 202 through the section 426.

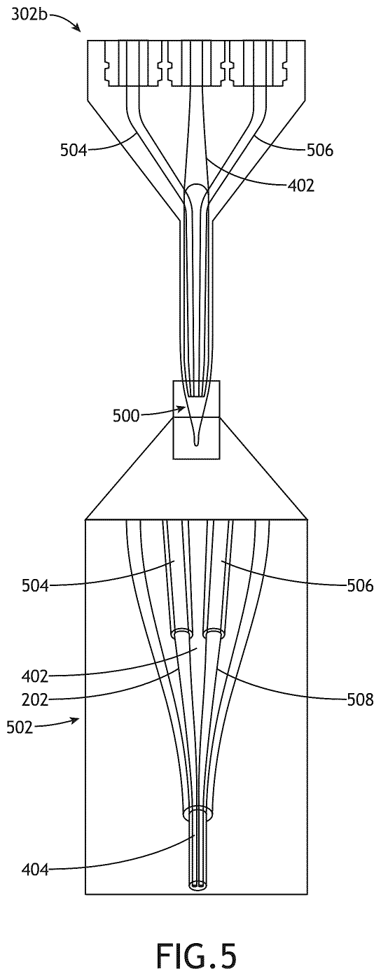

FIG. 5 illustrates a side view of an instrument head 302b, with a tip 500 of the instrument head 302b shown enlarged in an inset 502, in accordance with one or more embodiments of the disclosure. In embodiments, the instrument head 302b is configured to receive two illumination sources. For example, the instrument head 302b may include a first fiber 504 for propagating the transfer beam 202, and a second fiber 506 for propagating a photopolymerization beam 508. Instrument head 302b includes donor material path 402. The transfer beam 202, photopolymerization beam 508 and donor material 102 moving through the donor material path 402 converge at or near the aperture 404. The photopolymerization beam 508 photopolymerizes photopolymerizable materials within the donor material 102 such as collagen and hydrogels, as they are being printed onto the receiver material 104.

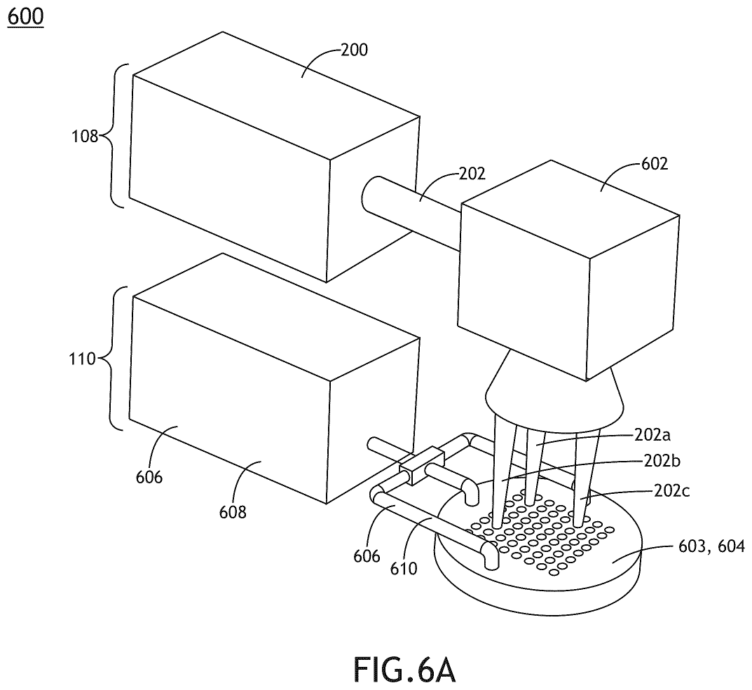

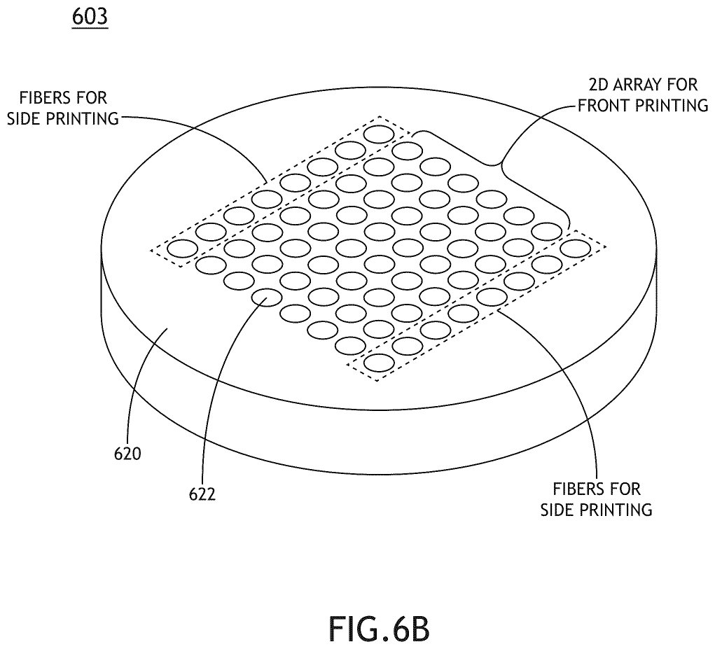

FIG. 6A illustrates a system 600 for printing donor material 102 onto receiver material 104 in accordance with one or more embodiments of the disclosure. System 600 may include one or more components of system 100 and vice versa. For example, the 600 may include a material transfer sub-system 108 and a donor supply sub-system 110. The material transfer sub-system 108 may include a transfer laser 200 propagating a transfer beam 202, and a beam scanner head 602 (e.g., a galvanometric scanning head) configured to receive the transfer beam 202 and direct and focus the transfer beam onto an input 603 of an optical fiber array 604 (e.g., a 2D optical fiber array with printing capabilities). The donor supply sub-system 110 may include a donor supply feeder system 606 comprising a reservoir 608 and tubing 610 that lead to an optical fiber array 604. Once the donor supply feeder system 606 feeds donor material 102 into the optical fiber array 604, the transfer beam 202a, 202b, 202c directs the donor material towards the receiver material 104 (e.g., in a LIFT-directed manner).

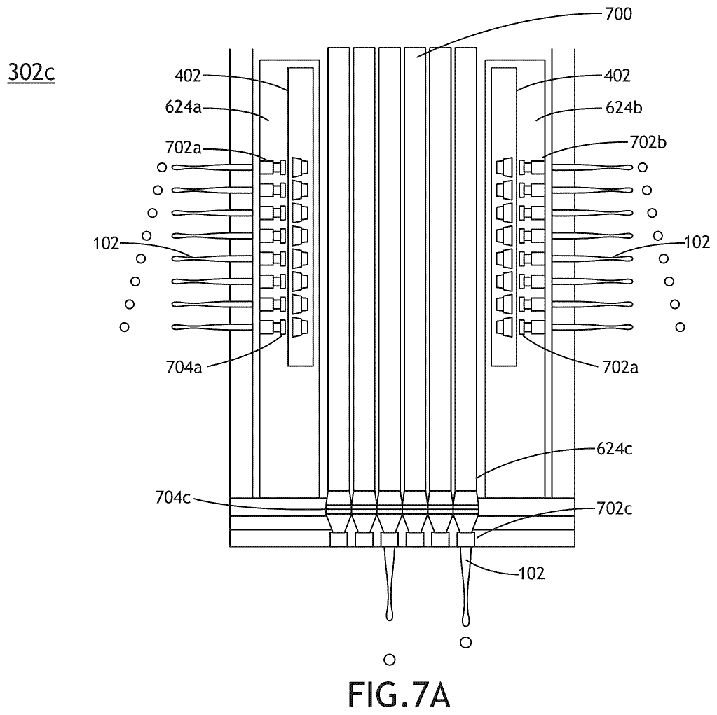

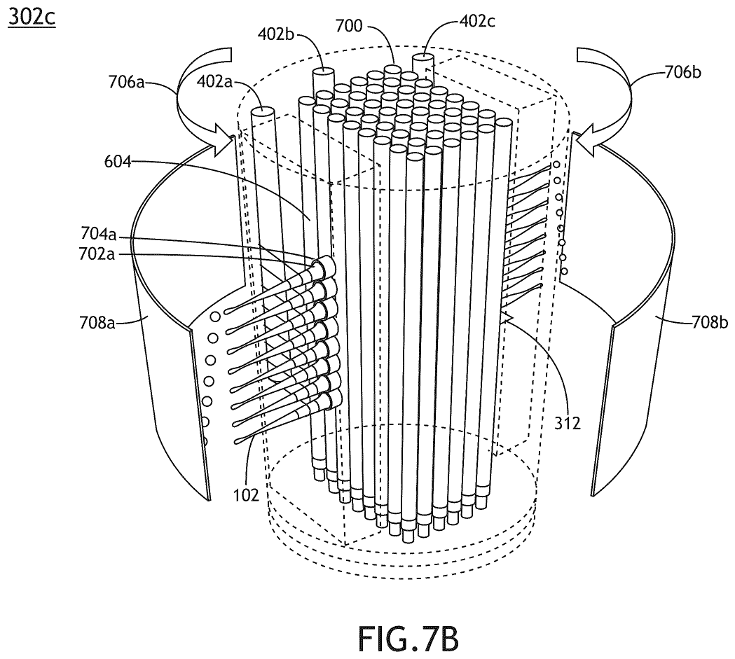

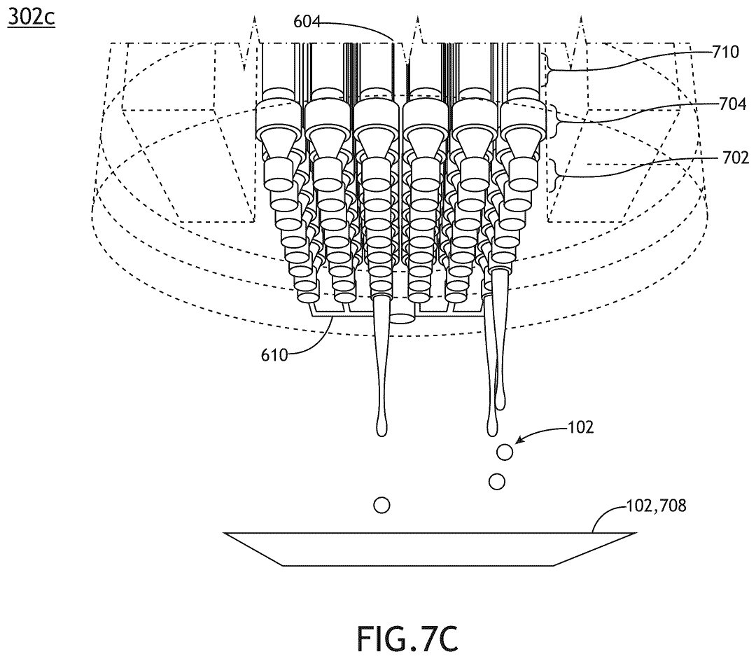

FIG. 6B illustrates a perspective view of the input 603 of the optical fiber array 604, in accordance with one or more embodiments of the disclosure. The view has been magnified to more clearly illustrate the ability of the instrument head 302c to direct the transfer beam 202 through specific optical fibers of the optical fiber array 604 and whether the printing of donor material 102 is performed from either the front of the instrument head 302c or from the sides of the instrument head 302c. The decision to print from the front or side of the instrument head 302c may be made by selecting the appropriate fibers in the optical fiber array 604, utilizing the beam scanner head 602. The donor feeder system 606 supplies donor material 102 through tubing 610 to micro-reservoirs located on the sides of the instrument head 302c, as shown in FIGS. 7A-7C. The input 603 may include a base 620 supporting one side of the optical fiber array 604 and apertures 622 for the optical fiber array 604.

FIG. 7A-7C illustrate views of an instrument head 302c, in accordance with one or more embodiments of the disclosure. The instrument head 302c may include one or more components of instrument heads 302a, 302b, and vice versa.

In embodiments, the instrument head 302c includes one or more optical fibers 700 or fiber arrays, as shown in FIG. 7A. For example, the instrument head 302c may include one or more optical fiber array subsets 624a, 624b, 624c that branch away from the main optical fiber array 604. Individual fibers of the optical array 604 for side printing and/or printing at the front section of the instrument head 302c. For example, the instrument head 302c may include two or more optical fiber array subsets 624 arranged orthogonal to each other (e.g., side and bottom) and/or opposite to each other. Donor material 102 is fed to the one or more sets of micro-reservoirs 702a, 702b, 702c, via a donor material path 402, resulting in the formation of microfluidic pools that await transfer by the transfer beam 202 directed by the one or more optical fibers 700.

For example, for front side printing, each fiber output will be optically coupled with a set of focusing lenses 704c of a lens array arrangement. The dimensions of the lens array will fit those of the optical fiber array 604. A microfluidic chip with cylindrical pools (e.g., micro-reservoir 702) under each fiber output-coupled focus lens 704 will be used as a donor. The volume of each pool in each micro-reservoir 702 will be approximately equal to the desired printed volume per droplet (some picolitres) or greater for multi-shot per pool printing mode. Similarly, the arrangement for the sideway printing will include sets of focusing lenses 704a, 704b coupled with the output of the fibers (e.g., from fiber array subsets 624a, 624b) and micro-reservoirs 702a, 702b under each fiber output. A small mirror or prism is mounted at the optical fiber tip to direct the laser beam toward the sidewalls. Alternatively, the fiber 700 is cut and/or polished in an angle to direct the laser beam sideways. To maintain the printing distance between donor and tissue, the arrangement may include micro-reservoirs 702a, 702b in a linear configuration (1D).

FIG. 7B illustrates a perspective view of the instrument head 302c, in accordance with one or more embodiments of the disclosure. In embodiments, at least a portion of the instrument head 302c is configured to rotate relative to the medical instrument 300 (e.g., the handle 306 of the medical instrument). Arrows 706a, 706b illustrate the movement relative of the instrument head 302c, enabling the instrument head to deliver donor material 102 (e.g., drops of fluid material) to form wide swaths 708a, 708b of deposited donor material 102.

FIG. 7C illustrates a perspective bottom view of the instrument head 302c depicting the optical fiber array 604 depositing donor material 102, forming a swath 708 of donor material 102. Separate micro-reservoirs 702a within the optical fiber array 604c are filled with microfluidic pools of donor material 102 via tubing coupled to the donor material path 402. The incoming transfer beam 202 enters the optical fiber array 604 and passes through an array of focusing lenses 704, before arriving at the array of micro-reservoirs 702 containing the microfluidic pools of donor material.

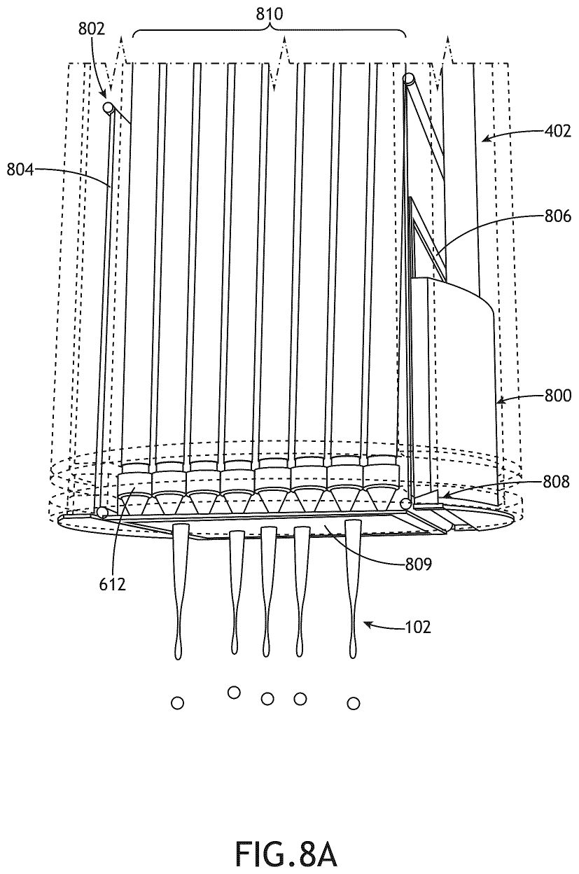

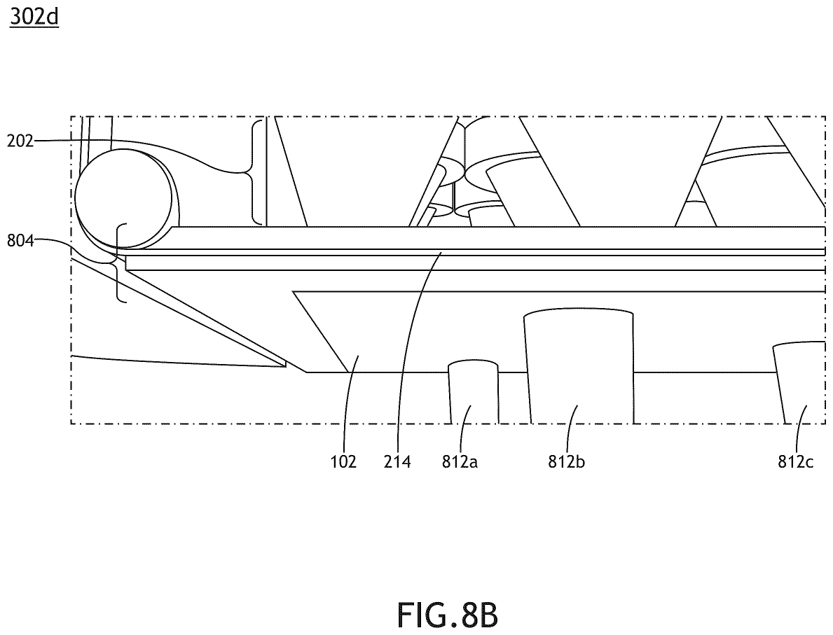

FIG. 8A illustrates a perspective front view of an instrument head 302d, in accordance with one or more embodiments of the disclosure. In embodiments, the instrument head 302d includes a donor material path 402 that leads to a donor material tank 800. The instrument head may further include a film rolling sub-system 802 that includes a film 804 that is threaded through a donor coating pathway 806. The film 804 contacts the donor material 102 from the donor material tank 800 at a film coating interface 808 (e.g., the donor material tank 800 fluidly coupled to the donor supply sub-system 110 and configured to supply donor material to the film coating interface 808). Film 804 coated with the donor material 102 is then moved to a position adjacent to the optical fiber array 604 at a print window 809. Transfer beams 202 propagating through an array of optical fibers 810, illuminates the film 804 coated with the donor material 102, cause the donor material 102 to eject.

FIG. 8B illustrates a close-up perspective view of an underside of the instrument head 302d, in accordance with one or more embodiments of the disclosure. In embodiments, transfer beams 202 from the transfer laser 200 illuminates the film 804 coated with donor material 102 (e.g., bioink), driving the donor material 102 toward the receiver material 104 (shown as droplets 812a, 812b, 812c).

In embodiment, the film 804 includes a laser absorbing layer 214 (e.g., gold sublayer) that absorbs energy from the transfer beam 202 and releases heat to the pooled donor material 102, causing the material transfer. In embodiments, the donor material 102 itself includes laser-absorbing material (e.g., blood).

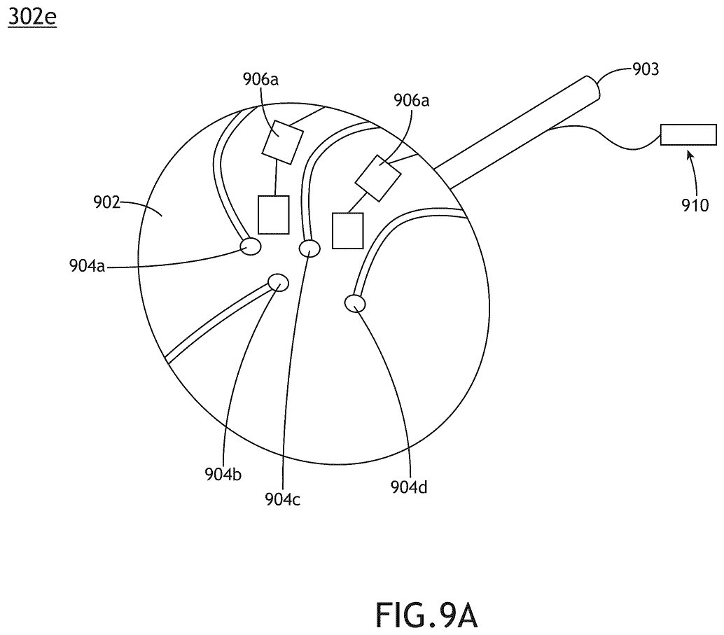

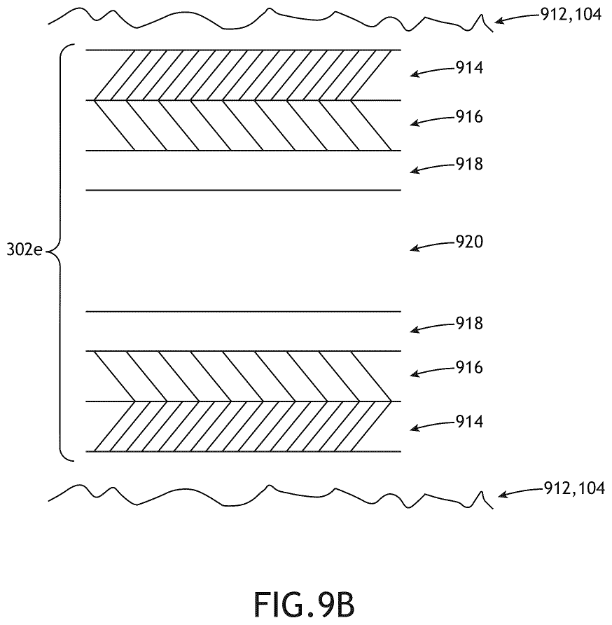

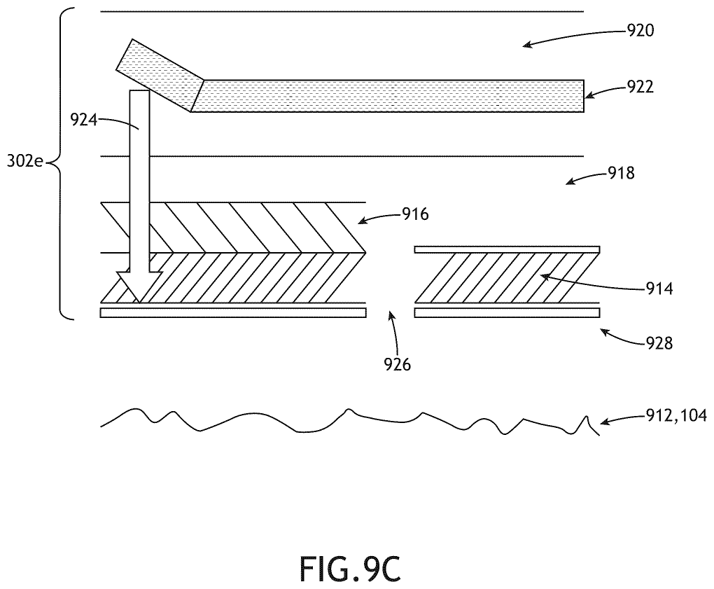

FIGS. 9A to 9C illustrate an instrument head 302e of a balloon type, in accordance with one or more embodiments of the disclosure. The instrument head 302e may include componentry for printing donor material based on one of several technologies including, but not limited to, LIFT. For example, the instrument head 302e may include one or more components of the instrument head 302a, 302b, 302c, 302d.

In embodiments, the instrument head 302e includes a printing structure 902 coupled to a stalk 903. The stalk 903 couples the printing structure to the donor supply sub-system 110 and/or the material transfer sub-system 108.

The delivery structure 902 may be of any shape. For example, the delivery structure 902 cross-section may not be uniform and include access locations for the transfer beam 202 or other components required for delivery of donor material 102. For instance, the delivery structure 902 may have a general appearance and shape of a balloon (e.g., comprises a balloon structure), such as a balloon used in angioplasty. In another instance, the printing structure 902 may have a general shape of a sphere.

The surface of the printing structure may include one or more fluidic connections 904a-d, and/or one or more electrical connections 906a-b. The one or more fluidic connections 904a-d and the one or more electrical connections 906a-b may enable the function of the instrument head 302e by providing electrical, sensor, and/or control support to the printing structure 902. For example, the one or more electrical connections 906a-b may supply power for donor material delivery and/or donor material printing systems in the printing structure. In another example, the one or more fluidic connections 904a-d may enable the transport of the donor material 102 with the instrument head 302e. For instance, the fluidic connections 904a-d may include microwells or delivery openings to deliver the donor material 102 (e.g., bioink and cells) to be printed.

The stalk 903 may tether the printing structure 902 to other control componentry of the system 100, such as a controller 112 or other control device that delivers one or more of electrical power (e.g., via an electrical source 910), transfer beams 202, or guidance. For example, the instrument head 302e may be steerable or articulated in many directions as needed for cell printing.

The materials and surfaces of the printing structure 902 may include componentry similar to the instrument heads 302 detailed above. For example, the printing structure 902 may include coatings that include polyimide and/or gold. The surface of the balloon may include other features such as microwells or delivery openings to deliver the bioink and cells to be printed. Balloons are used ubiquitously for minimally invasive procedures. Many balloons are therapeutic with electrical leads for atrial fibrillation balloon (AFB) therapy or other types of therapies. The printing device may include a bioprinting device that is internal to the balloon and can be steerable or articulated in many directions including 360-degree turning.

FIG. 9B illustrates a cross-section of the printing structure 902 (e.g., a balloon) in accordance with one or more embodiments of the disclosure. The printing structure 902 is shown positioned between two tissue surfaces 912 that may be designated as receiver material 104 for the printing of donor material 102 by the printing structure 902. For example, the two tissue surfaces 912 may include the inner surface of a blood vessel. The printing structure 902 may include a biocompatible outer surface 914 (e.g., acting as a donor substrate 210) made of biocompatible material including, but not limited to polyimide, gold, or Pebax.

In embodiments, printing structure 902 includes a sensor layer 916 that includes electronics (e.g., flexible electronic components), sensors, therapeutic leads, and other componentry. For example, the sensor layer 916 may include the one or more electrical connections 906a-b.

In embodiments, the printing structure 902 may further include a fluidics layer 918 for the storage, transport, and/or delivery of donor material. For example, the fluidics layer 918 may include the one or more fluidic connections 904a-d and capable of storing, at least temporarily, donor material 102. The printing structure 902 may further include an interior space 920 filled with air or fluid (e.g., water or saline) to inflate the printing structure 902.

FIG. 9C illustrates a close-up view of one-half of the cross-section of the printing structure 902 in accordance with one or more embodiments of the disclosure. The view shows more detail in the printing structure than as depicted in FIG. 9B. In embodiments, the printing structure 902 includes an energy conduit 922 configured to deliver energy (e.g., the transfer beam 202) to the printing structure 902. Once delivered to the printing structure 902, the transfer beam 202 may be distributed, as indicated by arrow 924. The delivery of laser light may be focused and steerable.

In embodiments, the printing structure 902 may include one or more openings 926 in the 928 and/or the sensor layer 916. The one or more openings 926 may enable transport of material (e.g., donor material 102) as well as enable electronic components within the sensor layer 916 to take measurements and/or transmit data. The printing structure 902 may also include a laser absorbing layer 214 (e.g., a gold coating) for use as a DRL.

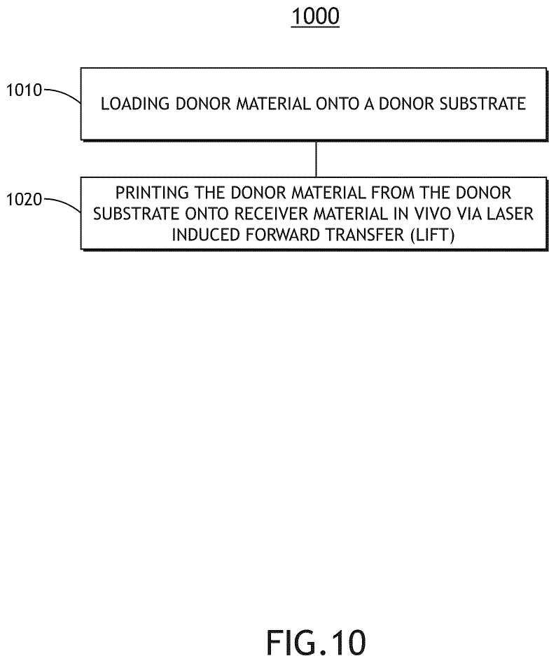

FIG. 10 illustrates a process flow diagram depicting a method 1000 for in-vivo cell replacement, in accordance with one or more embodiments of the disclosure. The method 1000 may be performed via any of the systems and devices as described herein.

In embodiments, the method 1000 includes a step 1010 of loading donor material 102 onto a donor substrate 210. For example, donor material 102 (e.g., bioink, cells, hydrogens) may be loaded onto any instrument head 302 or instrument head 302e via one or more components designated as, or acting as, a donor substrate 210.

In embodiments, the method 1000 includes a step 1020 of printing the donor material 102 from the donor substrate 210 onto receiver material 104 in vivo via Laser Induced Forward Transfer (LIFT). For example, donor material 102 comprising a mixture of cardiac compatible components (e.g., hydrogels, cardiomyocytes, extracellular matrix proteins, that have been loaded onto the donor substrate 210 (e.g., of the instrument head 302 or instrument head 302e) may be printed in vivo onto receiver material 104 (e.g., cardiac tissue) in an attempt to repair the underlying cardiac tissue.

Embodiments of the present disclosure will be described in the following examples, which are illustrative in nature.

### Example 1: Myocardial Thinning Restoration. Replacing Scar Tissue of the Myocardium

Myocardial thinning, a condition where the walls of the heart muscle (myocardium) become thinner, can be associated with various underlying medical conditions such as coronary artery disease (CAD), Myocardial Infarction, Chronic Myocarditis and genetic factors. After infarction, infarct thinning remains a consistent and prominent feature of geometric remodeling. Regardless of species and measurement technique, studies have reported progressive scar thinning to an average of ˜60% of initial thickness over several months and in some studies to as low as 20% While the central importance of scar structure in determining pump function and remodeling has long been recognized, it has proven remarkably difficult to design therapies that improve heart function or limit remodeling by modifying scar structure⁷ and while advancements in treating heart infarction have been made the related mortality rates continue to be elevated⁸. This underscores the urgency for exploring alternative therapeutic strategies focused on promoting tissue regeneration and reinstating cardiac functionality, rather than merely addressing the symptoms of the condition. In the last decade, a serious effort has been made with stem cell-based therapies. However, the lack of control over the cells after injection, the low number retainability of the cells in the target area, and the frequent failure of the cells to mature to functional cardiomyocytes¹⁰ tend to suggest that stem cell therapies for heart failure have not shown any clinically significant benefits.

Recently, tissue engineering has emerged as a field that seeks to address these challenges by integrating techniques for stem cell differentiation and maturation with advanced materials and bioprinting technologies. The construction of intricate 3D tissues and organs has become achievable through the careful selection of suitable biomaterials, cell types, and growth factors. For instance, the use of tissue-engineered cardiac patches is seen as a promising treatment alternative for individuals who have experienced a myocardial infarction. These patches work combinedly to provide both mechanical support and biological functionality for the restoration of a damaged myocardium. However, most of the materials used for the supporting structures, such as Extracellular matrix (ECM)-based hydrogels, have weak mechanical properties, making it difficult to later stitch them on the organ, or show impaired tissue function. The prospect of in vivo bioprinting, such as the devices and methods for LIFT as disclosed above, offers a hopeful approach to overcome the aforementioned challenges. This technique involves the direct printing of bioinks within the damaged area to generate or mend living tissues in a clinical environment. Specifically concerning myocardial thinning, where most therapies prove to be challenging, targeted in vivo laser bioprinting technique, which has the ability to print cardiomyocytes directly on the thinning area of the myocardium in order to regenerate the muscle.

LIFT is a cutting-edge technology that enables the controlled transfer of material from a donor film to a receiver material through the use of laser pulses. The disclosed apparatus comprises a laser source, a donor film, and a receiver material arranged in a controlled environment. The laser source emits pulses of a predetermined wavelength and energy, which are focused on the donor film.

The donor film is composed of a transparent carrier, at the laser wavelength used, on which the material to be transferred is coated, and it is positioned in close proximity to the receiver material. The laser light incident on the donor film induces a rapid and localized increase in pressure, causing a small portion of the material to be ejected from the donor film towards the receiver material. This transfer process occurs with high precision, allowing for the deposition of materials with sub-micron accuracy.

For the absorption of the light energy on the donor substrate, different approaches have been proposed, utilizing additional Dynamic Release Layers (DRL), when the material to be transferred is transparent at the wavelength used, or proposing direct absorption from the material to be transferred by introducing in its volume the appropriate absorbing agent in order to improve its absorption at the laser wavelength used.

In this study, the donor substrate will be realized on the output edge of an endoscopic device giving the opportunity of depositing the desired material (in this invention, living cells) directly on living tissue for the regeneration of human organs like the heart. Three different approaches for the realization of endoscopic laser Bioprinting are described next.

Single Fiber approach with Piezoelectric actuating movement. Examples of a single fiber approach for LIFT is illustrated in FIGS. 4A-4B and is described below. In embodiments, the tip can be sub-mm diameter or in the range of 1 mm-15 mm with possible configurations at, 1.5 mm, 2 mm, 4 mm, 8 mm. The length of the tip can be 1 mm or in the range of 1 mm-10 mm, with possible configurations at 1.5 mm, 2 mm, 4 mm, and 8 mm. It is also possible to be greater than 10 mm. The fiber optic can be single-mode fiber or multimode. The fiber is mounted on a piezoelectric tube that, once electrically wired and powered with specific X and Y signals, drives the fiber to vibrate. At specific frequencies and geometries of the cantilevered fiber resonances occur. Fiber resonance could be approximately 10 kHz in arrangements of 100 Hz-100 kHz in some embodiments. At 10 kHz the rate matches the rep rate of commercially available source lasers (5-10 kHz). This may be preferred so there is no lost pulse of the laser and yield is 100% from laser to cell injection. A possible preference may also include as high a repetition rate as possible and as high a scan rate as possible in order to provide a high throughput bioprinting effect. Energy and Intensity delivered at the target location may match as necessary for LIFT or LIST effects and ejection and printing of cells.

In embodiments, light is projected from the cantilevered optical fiber while it is actuated at its first mode of mechanical resonance to create a scan. Two-dimensional (2D) scan patterns are generated using a piezoelectric tube actuator with quartered electrodes. A circular scan can be generated when the horizontal and vertical resonant vibrations of the scanning fiber have the same frequency, but are 90 degrees out of phase. With axially symmetric scanning fibers, actuating a fiber at its resonant frequency allows for large fields of view in two dimensions. Modulating the amplitude of the drive signal allows for the creation of a spiral scan pattern. The projected light is focused with a lens system onto the target tissue area. By changing the amplitude of the drive signal input into the piezo scanner, the size of the target area can be altered At 250 spirals at 30 fps and a size of approximately 1 mm diameter of the module, the target area that is addressed for bioprinting is approx. 1 mm in diameter at scanning of e.g. 30V of the piezo, or reduced to approximately 300 um at 10 V of the piezo. These are approximate numbers so to show the scale of driving forces, size of the area, and time to scan the area.

In one embodiment, a (polyimide) PI film coated with gold (Au) is placed in front of the lens system and is replenished by motion as the area is fully scanned (after e.g., 250 spirals are performed). The motion is approximately 1 mm/second for the PI film to move and be replenished by a new area for the next “frame” of scanning. This velocity of 1 mm/s is common for people in the art and for adapting to a mechanism to deliver this motion for a minimally invasive catheter device at its tip or its side. In a different embodiment of the same concept, when the bio-ink to be transferred can absorb efficiently the laser beam wavelength, the flexible PI/Au film can be replaced with a transparent protective surface like glass or a plastic sheet.

In embodiments, a small mirror or prism is mounted at the optical fiber tip. Alternatively, the fiber is cut and/or polished at an angle to direct the laser beam sideways. In embodiments, the lens system is also mounted sideways so the bioprinting takes effect on the side of the tip. The fluidic channel and substrate with gold are mounted sideways too as it is easy to conceive for those knowledgeable in the art.

In embodiments, the fluidic delivery is a tubing that runs parallel to the laser delivery tip instead of being concentric. In that case, the cross-section of the fluidic tubing is a disk. Multiple such tubing can be used or attached to the laser scanning tip so they can deliver the same or multiple cell types at different times and locations for the bioprinting of shapes and volumes of tissue that may include a variety of combinations. Mimicking an organ tissue with e.g. vessels or muscle cells and epithelial cells is one example of this process. Multiple materials (e.g., cells in bioinks) may be printed simultaneously or in sequence in order to create hybrid tissue including, but not limited to, muscle and neurons, and epithelial cells on top of the vasculature.

Direct Flow and Dual-Laser Instrument Head. In embodiments, methods of delivery of cells to bioprinting fluidic channels include: gravity methods such as bags (e.g., such as IV bags) that deliver cells in bioink or media to the inlet of the cell delivery of the bioprinting device; syringe pumps, peristaltic pumps, pressure control systems with or without flow switch, and manual syringes. Methods of delivering cells to a bioprinting location via the bioprinting fluidic channels may include a round cannula that is concentric with the laser and other mechanisms of the bioprinting process. The diameter of the round cannula can be either larger than the bioprinting mechanism e.g., outside the laser delivery or in another embodiment can be central to the device while laser and other optical mechanisms are outside the cannula. In the first case, the cross-sectional area of the cell and medical delivery is a donut while in the second case a disk.

In embodiments, the cells are steered towards the location of printing and that can happen using a surface that has an angle with the first path of the cells. That angle can be discrete (e.g., 30 degrees, 60 degrees or 90 degrees, or can be a continuous surface that has a bending radius (e.g., of 1 mm, 2 mm, 5 mm, or 10 mm).

In embodiments, steering the cells via a curved area can focus the cells and concentrate the cells, or in another embodiment, it can have the opposite effect of diluting the cells or spreading the cells on the donor location to be printed.

Multi-fiber array combined with galvanometric scanning head. In embodiments, scanning is achieved by a combination of a galvanometric scanning head with a fiber array configuration for the delivery of the laser beam pulses inside the printing area, as shown in FIGS. 6A-7C. In this way, we can exploit the high scanning speed of a galvanometric scanning head (m/s) to print endoscopically. The printed areas will be predetermined from the size of the fiber array, in the range of mm×mm, and there will be the capability of covering larger areas with transitional movement of the endoscope, motorized or manual.

The array may cover a specific area (e.g., in square millimeters) according to the total size of the endoscope. For thoracoscopic surgeries, the total size of the endoscopes used is 12 mm where the fiber can cover an area of 4 mm×4 mm. For intravascular applications, the size of the endoscope tip can vary by reducing the total printing size down to 1 mm.

Different fibers can be used for delivering the laser pulses at the front side of the endoscope tip or at its sideways. With this option, printing can be achieved in highly curved organ regions, as well as on vessel walls. Two different approaches are considered for the realization of the donor at the end of the endoscopic tip.

In embodiments, a microfluidic chip with micro-reservoirs of specific volume will be used as donor substrate. The volumes of these micro-reservoirs will be filled, using an appropriate feeder, with the material to be transferred. In this case, no DRL is used and thus, the Bioink must absorb the laser wavelength by itself. An appropriate biocompatible absorbing agent can be diluted in the bioink for this purpose. For example, blood can be used for the 532 nm laser wavelength. Other substances that are biocompatible and used in the food industry include various food dyes such as Allura Red AC (E129), Amaranth (E123), Carmoisine (E122), and Ponceau 4R (E124).

For optimized results, parameters such as the height of the micro-reservoirs, the formation of a negative meniscus at the edge of each pooled donor material (determined by the volume of the material inside the micro-reservoirs), and the rheological properties of the material must be carefully considered and tailored to each specific case. These parameters influence the stability of the induced liquid jet and the quality of the printed droplet. Additionally, they determine the range of laser fluence appropriate for optimized transfer and the velocities acquired during the transfer of the material.

Concerning the rheological properties of the material, a non-Newtonian (shear-thinning) behavior may be preferable for improving printing quality. Non-Newtonian and more viscous liquids produce more stable jets, avoiding “splashing” onto the receiver material and the formation of satellite droplets, even at the same energy levels, compared to Newtonian and less viscous liquids. The non-Newtonian behavior can be tuned and optimized for each application by adding biocompatible thickening agents in tunable percentages, inducing shear-thinning behavior in the liquid material. A widely used example is xanthan gum, a high-molecular-weight polysaccharide, which, at very low concentrations (0.1% w/v in DMEM), can improve jet stability and the transfer of the material.

In the case of blood, its non-Newtonian behavior is primarily attributed to its red blood cells (RBCs). RBCs also contribute to absorption at 532 nm through their hemoglobin content. Consequently, a good choice for a biocompatible absorbing agent could be a patient's own blood after a process in which only the red blood cells are retained in blood plasma. This solution can be further improved by adding extra absorbing agents, such as those mentioned above, to enhance the absorption coefficient or by adding biocompatible thickening agents to improve the non-Newtonian behavior of the solution.

Concerning the height of the micro-reservoirs, it was observed that values approximately twice the diameter of the micro-reservoirs may be preferable for optimized printing results, ensuring a more stable liquid jet and reducing the formation of satellite droplets on the receiver.

Additionally, ensuring that the volume used for each droplet to fill the micro-reservoirs is appropriate for creating a negative meniscus at the surface of the pooled donor material results in improved printing quality. For that reason, a mechanism that can control the volume inserted into the micro-reservoirs in relation to the repetition rate of the laser is necessary to manage the volume used for printing each droplet. This mechanism can be implemented using a pump with a specific flow rate over time.

At the front side printing, each fiber output will be optically coupled with a focusing lens of a lens array arrangement. The dimensions of the lens array will fit those of the fiber array. A microfluidic chip with cylindrical micro-reservoirs under each fiber output-coupled focus lens will be used as a donor. The volume of each micro-reservoirs will be equal to the desired printed volume per droplet (some picolitres) or greater for multi-shot per pool printing mode.

In the same manner, the arrangement for the sideway printing may include focus lens arrays coupled with the output of the fibers and cylindrical micro-reservoirs under each fiber output. A small mirror or Prism is mounted at the optical fiber tip to direct the laser beam toward the sidewalls. Alternatively, the fiber is cut and/or polished at an angle to direct the laser beam sideways. In this case, in order to maintain the printing distance between donor and tissue, the arrangement may include micro-reservoirs in a linear configuration (1D). The 2D printing result is realized with rotation (motorized) of the endoscopic tip. Two or more outputs at specific angles on the sidewall of the tip can be used depending on the maximum angle of turn that will be able to be applied to the endoscopic tip. Simultaneous or independent printing from each sidewall output will be optional to cover different desired printing areas. A choice between the methods mentioned herein will be used to pump the bioink in order to fill the micro-reservoirs.

In embodiments, a Flexible DRL tank patterned donor configuration is used at the endoscopic tip as donor substrate. Here, the DRL layer absorbs the light energy, and the bioink can be transparent to the laser wavelength. The feeding of the donor is realized with methods like those described above and a tank. At the edge of the tank, a blade coater is used to spread homogenously the Bioink on the donor substrate. This spreading is realized by the movement of the donor film with a donor film rolling mechanism, and the volume is determined from the patterned tanks on the donor film. Front-side printing is enabled with this approach and different tips can be fabricated for sidewall printing in the same manner as mentioned in the second embodiment of the single fiber tip approach. Similarly, the dimensions of the printing area will be up to 5 mm×5 mm and larger areas will be achieved with transitional movement of the endoscope. The above-mentioned endoscopic tips can be fabricated separately to cover different types of printing needs, and they will all be able to fit onto a common endoscope probe for cell printing.

Photopolymerization at the Printing Area In an optional embodiment of the device, a method for the direct application of photopolymerization at the printing area has been implemented using either the same or a distinct endoscopic tool. 3D structures of printed cells, with a specific thickness (˜mm), are necessary to be constructed for applications like myocardial wall regeneration. To achieve this, cells are deposited diluted within liquid photopolymerized materials (like collagen and hydrogels). In another approach, extracellular matrices are fabricated to form 3D structures where the cells are being deposited. The materials used for these structures are biocompatible monomers or oligomers in a liquid state. They transform from liquid to solid by subjecting them to photopolymerization upon exposure to the light source of a specific wavelength, forming a robust three-dimensional structure. This particular embodiment enables the fabrication of such structures endoscopically and directly on the affected tissue, providing a targeted and minimally invasive approach.

In this embodiment, an additional light source emitting at a specific wavelength in the UV, VIS, or IR region of the spectrum is needed, suitable for the polymerization of the material of interest. In a different embodiment, a dual-band laser source can be used together with a beam-splitting configuration to separate the printing beam from the photopolymerization one.

The transmission of the polymerization beam into the printing area is facilitated using additional optical fibers compatible with the wavelength required for this process. These optical fibers can be positioned either at the center of the two-dimensional fiber array, intended for the printing function, or at various points in a coaxial and symmetrical arrangement concerning its center. The coupling of the beam at the input of these optical fibers is achieved either directly by mounting the fiber at the laser output or, in an alternative embodiment, through the utilization of the same scanning head employed for the printing process. In the latter case, the scanning head is equipped with dual-band mirrors for optimal performance in both wavelengths.

This specific embodiment of photopolymerization may be integrated into all endoscope configurations disclosed herein. In the single-fiber embodiments, additional fibers dedicated to photopolymerization surround the printing fiber. In the cylindrical micro-reservoirs-based donor endoscope embodiment, light for photopolymerization efficiently passes between the micro-reservoirs through the gaps formed between them. This approach ensures adequate illumination of the printing area. In the flexible donor film embodiment, specific areas on the donor film are modified to incorporate intentional openings or micro-reservoirs where the DRL layer is intentionally absent. These openings allow the photopolymerization light to pass, enabling precise and controlled polymerization on the printed structures.

Printing cell-laden bioinks in complex or less complex hydrogels. For heart repair, minimally invasive printing will be used with hydrogels that can be polymerizable with light, temperature, or other means and will be used in bioinks together with differentiated cardiomyocytes from autologous or immunocompatible iPS, or autologous mesenchymal stem cells from adipose tissue, or mesenchymal stem cells from the bone marrow, or any other cell type that can differentiate towards the cardiomyocyte fate. Hydrogels can be modified extracellular matrix proteins or autologous/heterologous platelet-rich plasma. The hydrogel can be further modified to contain growth factors that support cardiomyocyte growth, inhibit fibrosis, modulate inflammation, and suppress cell apoptosis. Depending on the anatomical site of cell printing, cells can be printed without the blood supply being interrupted (heart wall in infarction cases through the epicardium), or following blood slow down for inside the heart printing (through the endocardium). Hydrogels may be further modified to include multiple cell types such as cardiac fibroblasts, endothelial cells, or stem cells which can generate those cell types, in order to facilitate the integration and differentiation of cardiomyocytes and neovascularization of both the transplanted cells and damaged/diseased tissue under repair.

Another example may include, but is not limited to, a printing of pneumocytes for treating lung conditions such as chronic respiratory failure/chronic obstructive pulmonary disease or respiratory failure due to thoracic surgery (lobectomy) due to lung cancer. Another example could be hepatic failure. In both examples, functional augmentation of the target tissue by adding live cells (hepatocytes) which will integrate into the damaged or inadequately functioning tissue.

Another example is the printing of cells in any part of the GI tract, including esophageal cells for treating Barrett's esophagus or other conditions, the stomach, the small intestine, and the large intestine. Another example is the printing of urothelial cells for augmentation cystoplasty, orthotopic neobladder engineering, ileal conduit engineering ureteral and urethra repair.

Another example is the minimally invasive printing of chondrocytes for the repair of small injuries or more extensive damage of cartilage in joints. Bioprinting of neural cells, including glial cells and neurons in the central nervous system, including the brain and the spinal cord, may be performed to address a wide range of neurodegenerative conditions and trauma-related repair needs. Bioprinting may also include printing of any pancreatic subtype, in particular insulinogenic beta cells, or cells that can differentiate to produce insulin. For gynecological issues, including infertility issues, bioprinting may address these problems by printing uterine cells for the thickening of the uterine wall and improving the implantation behavior of the uterus.

Bioprinting may include printing skin cells to repair local damage such as scars, discoloration, and other skin conditions. Bioprinting may include printing of corneal and retinal cells to treat a number of eye conditions, including age-related macular degeneration, cataracts, diabetic retinopathy, and glaucoma. In general, printing of any cell type or hydrogel on any site in the body where access through natural or invasive routes can be achieved.

One or more processors 114 may include any type of processing element, including but not limited to integrated circuits (e.g., application-specific integrated circuits (ASIC) and field programmable gate arrays (FPGA). Memory 116 may also include resident or external memory for storing data, execution code, and other resident or external memory generated by the system 100. The controller 112 can execute one or more software programs embodied in a non-transitory computer-readable medium (e.g., memory 116) that implement techniques described herein. In some embodiments, the controller 112 is not limited by the materials from which it is formed or the processing mechanisms employed therein and, as such, can be implemented via semiconductor(s) and/or transistors (e.g., using electronic integrated circuit (IC) components), and so forth.

The memory 116 can be an example of tangible, computer-readable storage medium that provides storage functionality to store various data and/or program code associated with operation of the system 100 and/or controller 112, such as software programs and/or code segments, or other data to instruct the controller 112, and possibly other components of system 100, to perform the functionality described herein. Thus, the memory 116 can store data, such as a program of instructions for operating the main controller 112 and other components of the system 100. It should be noted that while a single memory 116 is described, a wide variety of types of memory 116 (e.g., tangible, non-transitory memory) may be employed. The 116 can be integral with the controller 112, can comprise stand-alone memory, or can be a combination of both. Some examples of the memory 116 may include removable and non-removable memory components, such as random-access memory (RAM), read-only memory (ROM), flash memory (e.g., a secure digital (SD) memory card, a mini-SD memory card, and/or a micro-SD memory card), solid-state drive (SSD) memory, magnetic memory, optical memory, universal serial bus (USB) memory devices, hard disk memory, external memory, and so forth.

What is claimed:

1. A system comprising:
a donor supply sub-system configured to supply donor material to a donor substrate;
a material transfer sub-system comprising a laser configured to illuminate the donor substrate via a transfer beam; and
an instrument head of a medical instrument configured to house the donor substrate, wherein upon an illumination of the donor substrate, the donor material is transferred to receiver material in vivo.

2. The system of claim 1, wherein the medical instrument comprises an endoscope.

3. The system of claim 1, wherein the instrument head comprises:
at least one optical fiber configured to propagate the transfer beam; and
a piezoelectric actuator coupled to the at least one optical fiber and configured to steer the at least one optical fiber.

4. The system of claim 3, wherein the instrument head further comprises:
an angled section; and
an optical element configured to direct the transfer beam through the angled section.

5. The system of claim 1, wherein the instrument head comprises a first fiber configured to propagate the transfer beam and a second fiber configured to propagate a photopolymerization beam.

6. The system of claim 1 comprising at least one optical fiber array, wherein the at least one optical fiber array comprises an array of micro-reservoirs configured to receive donor material, where upon illumination by the transfer beam, the donor material is transferred from the array of micro-reservoirs.

7. The system of claim 6, wherein the instrument head further comprises a fiber array configured to propagate the transfer beam to the array of micro-reservoirs.

8. The system of claim 6, wherein at least a portion of the instrument head is configured to rotate relative to the medical instrument.

9. The system of claim 6, wherein the instrument head comprises two optical fiber arrays arranged orthogonal relative to each other.

10. The system of claim 1, further comprising a film rolling sub-system comprising;
a donor material tank coupled to the donor supply sub-system;
a film coating interface fluidly coupled to the donor material tank; and
a film threaded within the film rolling sub-system and configured to receive a coating of donor material from the film coating interface, wherein upon movement of the film coated with the donor material to a print window, the film receives the transfer beam.

11. The system of claim 1, wherein the instrument head comprises:
a printing structure coupled to at least one of the donor supply sub-system and the material transfer sub-system, the printing structure comprising:
an outer surface;
a sensor layer comprising at least one sensor; and
a fluidics layer capable of storing donor material.

12. The system of claim 11, wherein the instrument head comprises a balloon structure.

13. The system of claim 1, further comprising at least one of a laser absorbing layer configured to absorb energy from the laser and release energy onto the donor material.

14. The system of claim 1 wherein the donor material includes a laser-absorbing agent.

15. A method for in-vivo cell replacement comprising:
loading donor material onto a donor substrate; and
printing the donor material from the donor substrate via an instrument head of a medical instrument onto receiver material in vivo via Laser Induced Forward Transfer (LIFT).

16. The method of claim 15, wherein the medical instrument comprises an endoscope.

17. The method of claim 15, wherein the instrument head comprises a balloon structure.

18. The method of claim 15, wherein the donor material comprises cells.

19. The method of claim 15, wherein the donor material further comprises at least one of hydrogels, growth factors, matrix proteins, and platelet-rich plasma.

20. The method of claim 18, wherein the cells comprise cardiac cells.
